(19) United States
(12) Patent Application Publication
(10) Pub. No.: US 2025/0352191 A1
(43) Pub. Date: Nov. 20, 2025

(54) FLUID APPLICATOR

(71) Applicant: Solventum Intellectual Properties Company, Maplewood, MN (US)

(72) Inventors: Dominic Nolan, Salisbury (GB); Alan R. Dombrowski, Woodbury, MN (US); Joseph Dartnell, Poole (GB); Iwen F. Grigsby, New Brighton, MN (US); Val Vossen, Minneapolis, MN (US); Semra Colak Atan, Edina, MN (US)

(21) Appl. No.: 18/872,331

(22) PCT Filed: Jun. 6, 2023
(86) PCT No.: PCT/IB2023/055815

§ 371 Date: Dec. 6, 2024

Related U.S. Application Data
(60) Provisional application No. 63/350,510, filed on Jun. 9, 2022.

(51) Int. Cl. A61B 17/00

(52) U.S. Cl. CPC A61B 17/00491; A61B 2017/00495

(57) ABSTRACT

A fluid applicator includes a main housing extending along a longitudinal axis defined between a first end and an opposing second end. The main housing defines an applicator outlet disposed at the first end. The fluid applicator further includes a first cylinder disposed within the main housing and including a first seal for holding a first fluid. The fluid applicator further includes a second cylinder spaced apart from the first cylinder and disposed within the main housing. The second cylinder includes a second seal for holding a second fluid different from the first fluid. The fluid applicator further includes a mixing chamber housing disposed within the main housing. The fluid applicator further includes a first piercing element, a second piercing element, and a nozzle connected to the mixing chamber housing opposite to each of the first piercing element and the second piercing element

### TECHNICAL FIELD

The present disclosure relates generally to a fluid applicator for dispensing or application of a fluid.

### BACKGROUND

Staples and sutures are commonly used for wound closure in operating room settings. While they are used for high tension and large closures, tissue adhesives alone or in combination with inner deep sutures are also used for small wounds. Tissue adhesives can be less painful, can lead to a better cosmetic outcome, and provide an antimicrobial barrier against further infections. As compared to staples and sutures, tissue adhesives can also be applied rapidly. Common tissue adhesives are cyanoacrylate-based compositions which have a very low viscosity. In addition, dispensers for these are often a sealed tube containing the tissue adhesive, where once the opening is unsealed, the tube is simply squeezed to deliver the low viscosity adhesive. Therefore, these commonly used tissue adhesives are not easily controlled upon delivery to a wound.

In order to rectify these issues, a two-part higher viscosity tissue adhesive and an applicator were developed. The applicator has been described in U.S. provisional application No. 63/350,510, filed Jun. 9, 2022, and the adhesive covered in WO 2022/090842 A1. The respective components priamine bis(oxamide monoethyl ester) and 2-ethylhexylglicidyl ether-modified PEI have viscosities of 8-13 Pascals-second and 21-26 Pascals-second. The applicator is designed to mix the two substantially different viscous components together (at a ratio of 1:1 equal masses) and dispense them through a nozzle with a static mixer, and then through a bead shaping tip to provide for easy application to the wound site.

Because the fluids are such different viscosities, technical challenges arose in obtaining good in line mixing and performance of the final dispense adhesive bead over the wound site. The dispensing and mixing problems that were discovered and solved are discussed in this application.

### SUMMARY

In a first aspect, the present disclosure provides a fluid applicator. The fluid applicator includes a main housing including a first end and a second end opposite to the first end. The main housing extends along a longitudinal axis defined between the first end and the second end. The main housing defines an applicator outlet disposed at the first end. The fluid applicator further includes a first cylinder disposed within the main housing and including a first seal for holding a first fluid. At least 85% of a total volume of the first cylinder is filled with the first fluid. The fluid applicator further includes a second cylinder spaced apart from the first cylinder and disposed within the main housing. The second cylinder includes a second seal for holding a second fluid different from the first fluid. At least 85% of a total volume of the second cylinder is filled with the second fluid. The fluid applicator further includes a mixing chamber housing disposed within the main housing and spaced apart from each of the first cylinder and the second cylinder along the longitudinal axis. The fluid applicator further includes a first piercing element substantially aligned with the first seal along the longitudinal axis and disposed between the mixing chamber housing and the first cylinder. The first piercing element includes a first cylindrical base connected to the mixing chamber housing and extending towards the first cylinder. The first piercing element further includes a first tip spaced apart from the mixing chamber housing and disposed proximal to the first seal. The first piercing element further includes a plurality of first walls extending from the first cylindrical base obliquely along the longitudinal axis. The plurality of first walls is angularly spaced apart from each other at the first cylindrical base and converge towards each other to intersect at the first tip. The fluid applicator further includes a second piercing element substantially aligned with the second seal along the longitudinal axis and disposed between the mixing chamber housing and the second cylinder. The second piercing element includes a second cylindrical base connected to the mixing chamber housing and extending towards the second cylinder. The second piercing element further includes a second tip spaced apart from the mixing chamber housing and disposed proximal to the second seal. The second piercing element further includes a plurality of second walls extending from the second cylindrical base obliquely along the longitudinal axis. The plurality of second walls is angularly spaced apart from each other at the second cylindrical base and converge towards each other to intersect at the second tip. The fluid applicator further includes a nozzle connected to the mixing chamber housing opposite to each of the first piercing element and the second piercing element. The nozzle extends from the mixing chamber housing to the applicator outlet. The nozzle and the mixing chamber housing define a mixing chamber cavity. Upon application of a force at the second end of the main housing, the first tip engages and pierces the first seal into a plurality of first flaps corresponding to the plurality of first walls, thereby creating a first fluid path between the first cylinder and the mixing chamber cavity. The first fluid flows through the first fluid path from the first cylinder to the mixing chamber cavity. Further, upon application of the force at the second end of the main housing, the second tip engages and pierces the second seal into a plurality of second flaps corresponding to the plurality of second walls, thereby creating a second fluid path between the second cylinder and the mixing chamber cavity. The second fluid flows through the second fluid path from the second cylinder to the mixing chamber cavity.

In a second aspect, the present disclosure provides a fluid applicator. The fluid applicator includes a main housing including a first end and a second end opposite to the first end. The main housing extends along a longitudinal axis defined between the first end and the second end. The main housing defines an applicator outlet disposed at the first end. The fluid applicator further includes a first cylinder disposed within the main housing and including a first seal for holding a first fluid. The fluid applicator further includes a second cylinder spaced apart from the first cylinder and disposed within the main housing. The second cylinder includes a second seal for holding a second fluid different from the first fluid. The fluid applicator further includes a mixing chamber housing disposed within the main housing and spaced apart from each of the first cylinder and the second cylinder along the longitudinal axis. The fluid applicator further includes a first piercing element substantially aligned with the first seal along the longitudinal axis and disposed between the mixing chamber housing and the first cylinder. The fluid applicator further includes a second piercing element substantially aligned with the second seal along the longitudinal axis and disposed between the mixing chamber housing and the second cylinder. The fluid applicator further includes a nozzle connected to the mixing chamber housing opposite to each of the first piercing element and the second piercing element. The nozzle extends from the mixing chamber housing to the applicator outlet. The nozzle and the mixing chamber housing define a mixing chamber cavity. Upon application of a force at the second end of the main housing, the first piercing element engages and pierces the first seal, thereby creating a first fluid path between the first cylinder and the mixing chamber cavity. The first fluid flows through the first fluid path from the first cylinder to the mixing chamber cavity. Further, upon application of the force at the second end of the main housing, the second piercing element engages and pierces the second seal, thereby creating a second fluid path between the second cylinder and the mixing chamber cavity. The second fluid flows through the second fluid path from the second cylinder to the mixing chamber cavity. The fluid applicator further includes a nozzle gasket disposed inside the mixing chamber housing and forming a sealing contact between the mixing chamber housing and the nozzle. The nozzle gasket defines a first through aperture in fluid communication with the first fluid path. The nozzle gasket further defines a second through aperture in fluid communication with the second fluid path. The nozzle gasket further defines a third through aperture disposed between the first through aperture and the second through aperture along a transverse axis perpendicular to the longitudinal axis, such that a portion of at least one of the first and second fluids which flows from the respective first and second fluid paths is trapped within the third through aperture.

In a third aspect, the present disclosure provides a fluid applicator. The fluid applicator includes a main housing including a first end and a second end opposite to the first end. The main housing extends along a longitudinal axis defined between the first end and the second end. The main housing defines an applicator outlet disposed at the first end. The applicator outlet has a substantially rectangular cross-section or an elliptical cross-section. The fluid applicator further includes a first cylinder disposed within the main housing and including a first seal for holding a first fluid. The fluid applicator further includes a second cylinder spaced apart from the first cylinder and disposed within the main housing. The second cylinder includes a second seal for holding a second fluid different from the first fluid. The fluid applicator further includes a mixing chamber housing disposed within the main housing and spaced apart from each of the first cylinder and the second cylinder along the longitudinal axis. The fluid applicator further includes a first piercing element substantially aligned with the first seal along the longitudinal axis and disposed between the mixing chamber housing and the first cylinder. The fluid applicator further includes a second piercing element substantially aligned with the second seal along the longitudinal axis and disposed between the mixing chamber housing and the second cylinder. The fluid applicator further includes a nozzle connected to the mixing chamber housing opposite to each of the first piercing element and the second piercing element. The nozzle extends from the mixing chamber housing to the applicator outlet. The nozzle and the mixing chamber housing define a mixing chamber cavity. Upon application of a force at the second end of the main housing, the first piercing element engages and pierces the first seal, thereby creating a first fluid path between the first cylinder and the mixing chamber cavity. The first fluid flows through the first fluid path from the first cylinder to the mixing chamber cavity. Further, upon application of the force at the second end of the main housing, the second piercing element engages and pierces the second seal, thereby creating a second fluid path between the second cylinder and the mixing chamber cavity. The second fluid flows through the second fluid path from the second cylinder to the mixing chamber cavity.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is an underside perspective view of a fluid applicator, according to an embodiment of the present disclosure;

FIG. 2 is an underside perspective view of the fluid applicator of FIG. 1, with some components partially shown, according to an embodiment of the present disclosure;

FIG. 3 is a perspective sectional side view of the fluid applicator of FIG. 1, according to an embodiment of the present disclosure;

FIG. 4 is a partial exploded view of the fluid applicator of FIG. 1, according to an embodiment of the present disclosure;

FIG. 5A is a side view of a mixing chamber housing, a first piercing element, and a second piercing element of the fluid applicator of FIG. 1, according to an embodiment of the present disclosure;

FIG. 5B is a top view of the mixing chamber housing and the first piercing element, according to an embodiment of the present disclosure;

FIG. 5C is a sectional side view of the mixing chamber housing, the first piercing element, and the second piercing element, according to an embodiment of the present disclosure;

FIG. 5D is a front view of the mixing chamber housing, the first piercing element, and the second piercing element, according to an embodiment of the present disclosure;

FIG. 5E is a perspective front view of the mixing chamber housing, the first piercing element, and the second piercing element, according to an embodiment of the present disclosure;

FIG. 6A is a front view of a pierced first seal in the fluid applicator of FIG. 1, according to an embodiment of the present disclosure;

FIG. 6B is a front view of a pierced second seal in the fluid applicator of FIG. 1, according to an embodiment of the present disclosure;

FIG. 7 is a sectional side view of the mixing chamber housing and a nozzle of the fluid applicator of FIG. 1, according to an embodiment of the present disclosure;

FIG. 8 is a front view of the nozzle of FIG. 7, according to an embodiment of the present disclosure;

FIG. 9 is a perspective front view of the nozzle of FIG. 7, according to an embodiment of the present disclosure;

FIG. 10 is a perspective sectional front view of the mixing chamber housing and the nozzle of FIG. 7, with some components not shown, according to an embodiment of the present disclosure;

FIG. 11 is a front view of the mixing chamber housing, the nozzle, and a nozzle gasket of the fluid applicator of FIG. 1, with other components not shown, according to an embodiment of the present disclosure;

FIG. 12 is a front view of the nozzle gasket of FIG. 11, according to an embodiment of the present disclosure;

FIG. 13 is a cross-sectional view of a first end of the fluid applicator of FIG. 1, according to an embodiment of the present disclosure;

FIG. 14 is a front view of the first end of FIG. 13, according to an embodiment of the present disclosure;

FIG. 15 is a rear view of the first end of FIG. 13, according to an embodiment of the present disclosure; and

FIG. 16 is another cross-sectional view of the first end of FIG. 13, according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, when a first material is termed as “same” or “similar” as a second material, at least 90 weight % of the first and second materials are identical and any variation between the first and second materials comprises less than about 10 weight % of each of the first and second materials.

As used herein, “at least one of A and B” should be understood to mean “only A, only B, or both A and B”.

Unless specified or limited otherwise, the terms “attached,” “connected,” “coupled”, and variations thereof, are used broadly and encompass both direct physical connections, or indirect physical connections between two or more components that are connected together by one or more additional components. For example, a first component may be coupled to a second component by being directly connected together or by being connected by a third component. In some examples, coupling, connection, and attachment may also include mechanical, thermal, electrical, or chemical coupling (such as a chemical bond) in some contexts.

As used herein, the term “wounds” may include, for example, chronic, acute, traumatic, subacute, closed surgical wounds or dehiscence wounds, partially thick burns, ulcers (such as, diabetic, compressive, or venous insufficiency ulcers), flaps, and grafts. The wound may also include an open abdomen area of a patient.

Tissue adhesives alone or in combination with inner deep sutures are used to heal and/or seal small wounds. Common tissue adhesives are cyanoacrylate-based compositions which have a very low viscosity. Applicators for delivering the tissue adhesives include sealed tube type dispensers which may not deliver a desirable amount of the tissue adhesives due to very low viscosity of the cyanoacrylate-based compositions in the tissue adhesives. By mixing two substantially different viscous components together, the viscosity of the tissue adhesive may be increased, however, it could still be subject to technical challenges in obtaining good in-line mixing and performance of the final dispense adhesive bead over the wounds.

One of the challenges could be obtaining a bubble free dispensing operation from conventional fluid applicators or dispensers. Formation of large bubbles may lower the tensile strength at localized locations along the bead length. Generally, a bubble free dispense is obtained by filling both cylinders of the conventional fluid applicator to a top end before each cylinder is sealed with a foil seal. This may eliminate any head space between the foil seal and top of the fluid in the cylinder. However, when the completed filled cylinders were tested with conventional design of piercing elements, the foil seal may tear away from cylinder rims and float in the formulation of the tissue adhesive. The floating foil piece may fully or partially block mixing chamber inlets during the dispensing operation. This was rectified with a new pierce element design, which pierced the foil into multiple smaller foil segments, and not one large flap as by the conventional design of piercing elements. Having a pierce design that resulted in one large flap being pierced needed head space (i.e., between foil and top of fill line) in the cylinder to allow the flap to move while the foil was getting pierced. However, as stated earlier, having this gap resulted in head space and hence an underfilled cylinder with an air pocket in it.

Further, in conventional fluid applicators or dispensers with a two-part tissue adhesive of different viscosities, other problems can exist. For example, upon activation of the applicator and when the fluids first flow from the cylinders into the mixing chamber, a lower viscosity fluid can initially lead a higher viscous fluid through the mixing chamber. This manifests itself as having material that then moves through the nozzle that cannot be mixed with the other component. This will occur only during the initial time period when the fluid fronts first come together upon activation. This may be observed by a lower tensile strength of the initial portion or a leading edge of the dispense bead. This can be solved in a few different ways. One way is to have a well in the mixing chamber cavity region, which can trap the initial front of the fluids as they first come together in the mixing chamber cavity. A second way is to put a flow restrictor in the cavity, in the side of the lower viscosity fluid. This could be a protrusion from the nozzle. This may limit the initial flow of the lower viscosity fluid upon activation of the applicator. These first two approaches address the issue “pre” mix, that is before the fluid is introduced into the nozzle and through a static mixer. A third approach is “post” mix, i.e., after the fluids have gone through the nozzle/inline mixer assembly. In this approach an applicator tip, which is attached to the nozzle exit, has a tortuous path and a fluid trap in which the trap catches the initial flow of fluid. After the trap is filled with the initial portion of the dispensed fluid, the remaining mixed fluid flows out of the nozzle, and through the tip without further capture of any additional fluid.

Moreover, in the conventional fluid applicators, the selected tip geometry may not provide desirable or proper dispense bead height and width. Since the two fluids of different viscosities need to mix with an inline static mixer that fits into a nozzle that has a substantially round internal cross section, a tip that changes the shape from a round profile to a wide narrow dispense bead is also needed. In order to adequately cover a wound incision, a dispense bead width should ideally be around 10-13 millimeter (mm). Thus, a novel geometry is needed to convert the nozzle output into the properly shaped dispense bead which is put on the wound closure site. The dispense bead has a domed profile, typically 10-13 mm wide and 1-3 mm in height. Hence, the width is much greater than the height.

The present disclosure provides a fluid applicator. The fluid applicator includes a main housing including a first end and a second end opposite to the first end. The main housing extends along a longitudinal axis defined between the first end and the second end. The main housing defines an applicator outlet disposed at the first end. The fluid applicator further includes a first cylinder disposed within the main housing and including a first seal for holding a first fluid. At least 85% of a total volume of the first cylinder is filled with the first fluid. The fluid applicator further includes a second cylinder spaced apart from the first cylinder and disposed within the main housing. The second cylinder includes a second seal for holding a second fluid different from the first fluid. At least 85% of a total volume of the second cylinder is filled with the second fluid. The fluid applicator further includes a mixing chamber housing disposed within the main housing and spaced apart from each of the first cylinder and the second cylinder along the longitudinal axis. The fluid applicator further includes a first piercing element substantially aligned with the first seal along the longitudinal axis and disposed between the mixing chamber housing and the first cylinder. The first piercing element includes a first cylindrical base connected to the mixing chamber housing and extending towards the first cylinder. The first piercing element further includes a first tip spaced apart from the mixing chamber housing and disposed proximal to the first seal. The first piercing element further includes a plurality of first walls extending from the first cylindrical base obliquely along the longitudinal axis. The plurality of first walls is angularly spaced apart from each other at the first cylindrical base and converge towards each other to intersect at the first tip. The fluid applicator further includes a second piercing element substantially aligned with the second seal along the longitudinal axis and disposed between the mixing chamber housing and the second cylinder. The second piercing element includes a second cylindrical base connected to the mixing chamber housing and extending towards the second cylinder. The second piercing element further includes a second tip spaced apart from the mixing chamber housing and disposed proximal to the second seal. The second piercing element further includes a plurality of second walls extending from the second cylindrical base obliquely along the longitudinal axis. The plurality of second walls is angularly spaced apart from each other at the second cylindrical base and converge towards each other to intersect at the second tip. The fluid applicator further includes a nozzle connected to the mixing chamber housing opposite to each of the first piercing element and the second piercing element. The nozzle extends from the mixing chamber housing to the applicator outlet. The nozzle and the mixing chamber housing define a mixing chamber cavity. Upon application of a force at the second end of the main housing, the first tip engages and pierces the first seal into a plurality of first flaps corresponding to the plurality of first walls, thereby creating a first fluid path between the first cylinder and the mixing chamber cavity. The first fluid flows through the first fluid path from the first cylinder to the mixing chamber cavity. Further, upon application of the force at the second end of the main housing, the second tip engages and pierces the second seal into a plurality of second flaps corresponding to the plurality of second walls, thereby creating a second fluid path between the second cylinder and the mixing chamber cavity. The second fluid flows through the second fluid path from the second cylinder to the mixing chamber cavity.

As the first tip engages and pierces the first seal into the plurality of first flaps corresponding to the plurality of first walls, the plurality of first flaps remains attached to the first seal of the first cylinder. Similarly, as the second tip engages and pierces the second seal into the plurality of second flaps corresponding to the plurality of second walls, the plurality of second flaps remains attached to the second seal of the second cylinder. In other words, the plurality of first flaps and the plurality of second flaps are not detached from the respective first seal and the second seal upon piercing of the respective first seal and the second seal. Due to geometrical design of the first piercing element and the second piercing element, the plurality of first flaps remains attached to the first seal of the first cylinder, and the plurality of second flaps remain attached to the second seal of the second cylinder. Therefore, no part of the first seal tears away from the first cylinder and no part of the second seal tears away from the second cylinder. This may ensure that during dispensing operation by the fluid applicator of the present disclosure, components, such as the mixing chamber cavity, the nozzle, and the applicator outlet, may not be blocked by any torn away pieces of the first seal and the second seal, which was otherwise a challenge in the conventional fluid applicators/dispensers.

In some embodiments, the mixing chamber housing and the nozzle further define a first portion substantially aligned with the first piercing element along the longitudinal axis. The first fluid flows through the first fluid path from the first cylinder to the first portion. The mixing chamber housing and the nozzle further define a second portion substantially aligned with the second piercing element along the longitudinal axis. The second fluid flows through the second fluid path from the second cylinder to the second portion. The mixing chamber housing and the nozzle further define a mixing through aperture in fluid communication with the mixing chamber cavity disposed between the first portion and the second portion along a transverse axis orthogonal to the longitudinal axis. The first fluid flows through the first portion to the mixing through aperture. The second fluid flows through the second portion to the mixing through aperture. The mixing chamber housing and the nozzle further define a flow restrictor disposed on the nozzle and between the first portion and the mixing through aperture along the transverse axis and extending along a sagittal axis orthogonal to each of the transverse axis and the longitudinal axis, such that the flow restrictor restricts the flow of the first fluid from the first portion to the mixing through aperture.

The flow restrictor may slow down an initial dispense of the first fluid before the first fluid meets the second fluid in the mixing chamber cavity. In some embodiments, a first viscosity of the first fluid is different from a second viscosity of the second fluid. In some embodiments, a ratio between the second viscosity of the second fluid and the first viscosity of the first fluid is greater than or equal to 2. This means that the first fluid is less viscous than the second fluid. Therefore, for a desirable tensile strength of the initial portion of the dispense bead, it is required to limit the flow rate of the first fluid because of the relatively lower viscosity of the first fluid. Hence, the flow restrictor limits or restricts the flow of the first fluid from the first portion to the mixing through aperture and may therefore improve the tensile strength of the initial portion of the dispense bead formed by even mixing of the first fluid and the second fluid.

In some embodiments, the fluid applicator further includes a nozzle gasket disposed inside the mixing chamber housing and forming a sealing contact between the mixing chamber housing and the nozzle. The nozzle gasket defines a first through aperture in fluid communication with the first fluid path. The nozzle gasket further defines a second through aperture in fluid communication with the second fluid path. The nozzle gasket further defines a third through aperture disposed between the first through aperture and the second through aperture along a transverse axis perpendicular to the longitudinal axis, such that a portion of at least one of the first and second fluids which flows from the respective first and second fluid paths is trapped within the third through aperture. In some embodiments, a volume of the portion of the at least one of the first and second fluids trapped within the third through aperture is greater than or equal to about 5 mm³ and less than or equal to about 25 mm³.

Thus, the third through aperture acts as a fluid trap to capture the initial dispense bead (i.e., the portion of the at least one of the first and second fluids which flows from the respective first and second fluid paths). The inclusion of the third through aperture in the nozzle gasket may remove an initial material that cannot be mixed (e.g., the initial portion of a first fluid having a lower viscosity) to provide an adequate composition of the dispense bead. This may further improve the tensile strength of the dispense bead formed by even mixing of the first fluid and the second fluid.

In some embodiments, the nozzle further includes a nozzle body in fluid communication with the mixing chamber cavity. The nozzle further includes a static mixer disposed inside the nozzle body. The nozzle further includes a nozzle tip defining a nozzle outlet for dispensing at least one of the first and second fluids from the nozzle. The nozzle tip is connected to and extends from the nozzle body. The nozzle outlet has a substantially circular cross-section. In some embodiments, the main housing includes a capture well adjacent the applicator outlet, such that a portion of at least one of the first and second fluids which flows from the nozzle outlet is trapped within the capture well. In some embodiments, the portion of the at least one of the first and second fluids which is trapped within the capture well is greater than or equal to about 2% and less than or equal to about 15% of the total volume of the first and second fluids to be dispensed from the nozzle outlet.

The capture well acts as a fluid trap to capture the initial bolus of a mixture of the first fluid and the second fluid after being mixed within the nozzle body. Therefore, a desirable tensile strength of the dispense bead may be obtained by capturing the portion of the at least one of the first and second fluids within the capture well.

In some embodiments, the applicator outlet has a substantially rectangular cross-section or an elliptical cross-section. In some embodiments, the applicator outlet includes a maximum outlet width along a transverse axis orthogonal to the longitudinal axis and a maximum outlet thickness orthogonal to each of the transverse axis and the longitudinal axis. The maximum outlet width is greater than the maximum outlet thickness by a factor of at least 3. In some embodiments, a ratio between the maximum outlet width of the applicator outlet and a maximum outlet width of the nozzle outlet is greater than or equal to about 2 and less than or equal to about 5.

Such dimensions of the maximum outlet width and the maximum outlet thickness of the applicator outlet may provide a desirable height and width of the dispense bead formed by mixing the first fluid and the second fluid. Such dimensions of the applicator outlet may cause the dispensed bead to achieve a desirable profile shape after exiting through the nozzle.

In some embodiments, the outlet interface includes a fluid separator disposed between the nozzle outlet and the applicator outlet. At least a portion of the fluid separator proximal to the nozzle outlet tapers outwardly relative to the longitudinal axis in a flow direction of at least one of the first and second fluids such that the flow of the at least one of the first and second fluids from the nozzle outlet is directed outward towards edges of the applicator outlet. In some embodiments, the remaining portion of the fluid separator proximal to the applicator outlet tapers inwardly relative to the longitudinal axis in the flow direction of the at least one of the first and second fluids such that the flow of the at least one of the first and second fluids from the nozzle outlet is directed inward towards the center of the applicator outlet. Therefore, the fluid separator directs the flow of the at least one of the first and second fluids from the nozzle outlet towards the edges as well as the center of the applicator outlet. This direction of the flow of the at least one of the first and second fluids from the nozzle outlet to the applicator outlet may ensure that the at least one of the first and second fluids or the mixture of the first and second fluids is spread uniformly and may provide a desirable height and width of the dispense bead that exits out of the applicator outlet.

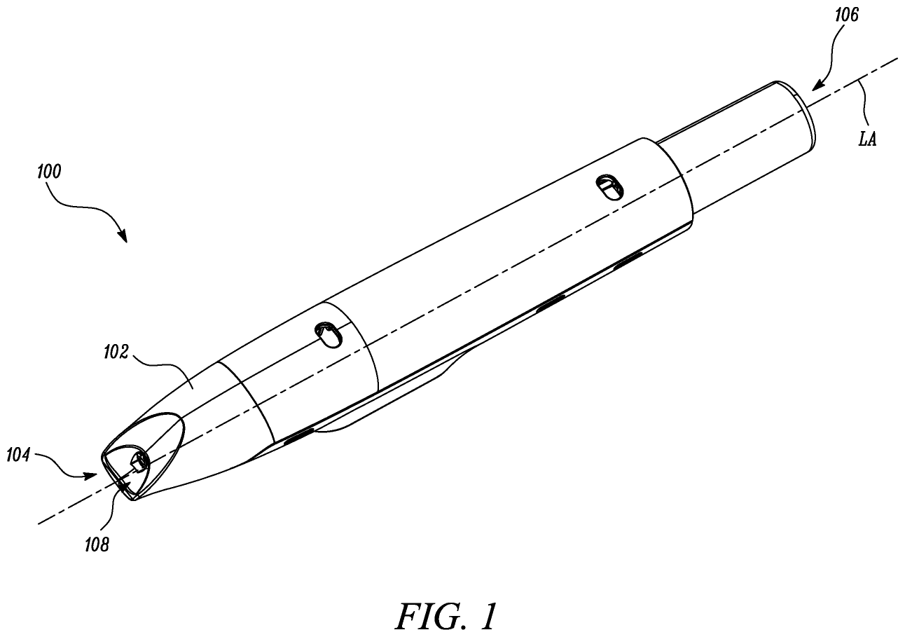

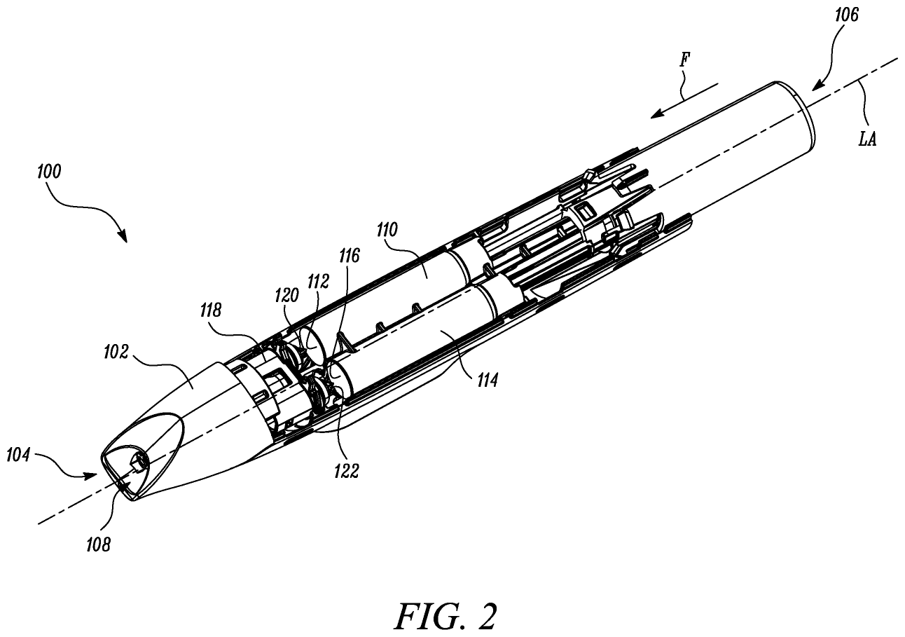

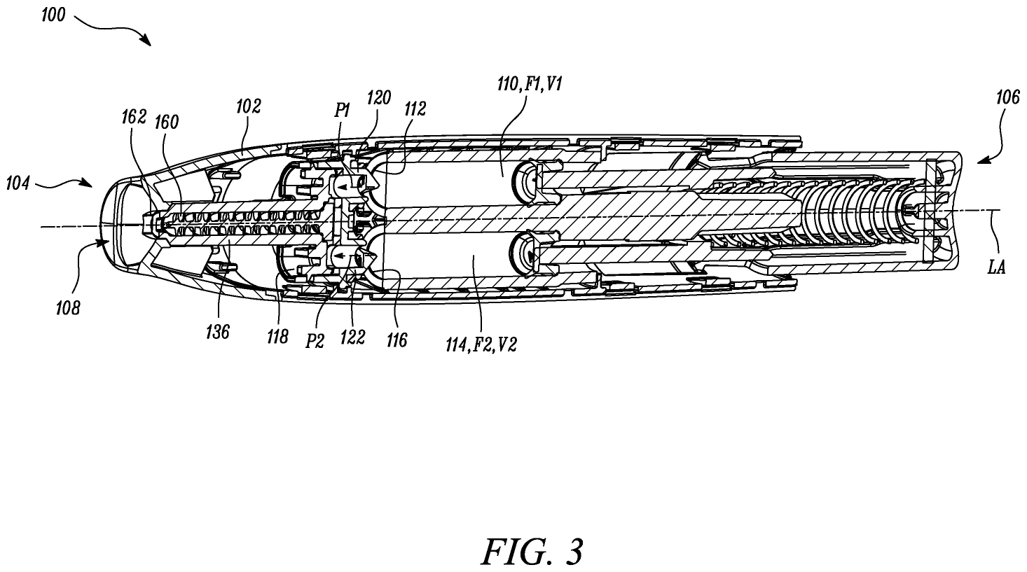

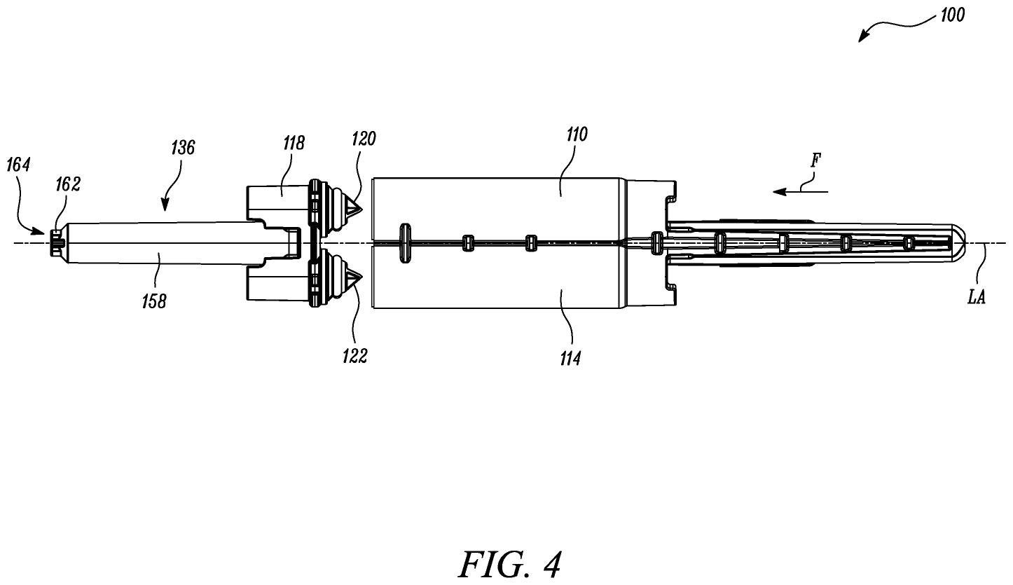

Referring now to Figures, FIG. 1 is an underside perspective view of a fluid applicator 100, according to an embodiment of the present disclosure. The fluid applicator 100 includes a main housing 102 that supports, holds, or contains some or all of the components of the fluid applicator 100. FIG. 2 is an underside perspective view of the fluid applicator 100, with some components partially shown, according to an embodiment of the present disclosure. Specifically, in FIG. 2, the main housing 102 is shown only partially. FIG. 3 is a sectional side view of the fluid applicator 100, according to an embodiment of the present disclosure. FIG. 4 is a partial exploded view of the fluid applicator 100, according to an embodiment of the present disclosure. Some components (such as the main housing 102) of the fluid applicator 100 are not shown in FIG. 4 for illustrative purposes. It should be noted that the fluid applicator 100 is illustrated in a deactivated configuration in FIGS. 1 to 4. In the deactivated configuration, the fluid applicator 100 cannot deliver or dispense any adhesive or fluid therefrom.

Referring to FIGS. 1 to 4, the main housing 102 includes a first end 104 and a second end 106 opposite to the first end 104. The main housing 102 extends along a longitudinal axis LA defined between the first end 104 and the second end 106. The main housing 102 defines an applicator outlet 108 disposed at the first end 104. The applicator outlet 108 is a component from which a fluid is dispensed or applied onto a surface (e.g., skin).

The fluid applicator 100 further includes a first cylinder 110 disposed within the main housing 102 and including a first seal 112 for holding a first fluid F1. At least 850% of a total volume of the first cylinder 110 is filled with the first fluid F1. In some other embodiments, at least 90%, or at least 95% of the total volume of the first cylinder 110 may be filled with the first fluid F1. In some embodiments, the first cylinder 110 may be fully filled with the first fluid F1. The first fluid F1 may be an adhesive, a skin protectant, oil, or solvent.

The fluid applicator 100 further includes a second cylinder 114 spaced apart from the first cylinder 110 and disposed within the main housing 102. The second cylinder includes a second seal 116 for holding a second fluid F2 different from the first fluid F1. At least 85% of a total volume of the second cylinder 114 is filled with the second fluid F2. In some other embodiments, at least 90%, or at least 95% of the total volume of the second cylinder 114 may be filled with the second fluid F2. In some embodiments, the second cylinder 114 may be fully filled with the second fluid F2. The second fluid F2 may be an adhesive, a skin protectant, oil, or solvent.

In some embodiments, the first fluid F1 and the second fluid F2 may be reactive with one another and therefore must be separated during storage, and in use upon mixing a change occurs. In some embodiments, the first fluid F1 and the second fluid F2 form a two-part adhesive, that crosslink together. An example of a two-part adhesive is epoxy. In some embodiments, the one of the first and second fluids F1, F2 contains an oxalamido-containing compound while the other of the first and second fluids F1, F2 contains a derivatized polyethylene imine, and the first fluid F1 and the second fluid F2 mix together to form a multiple-part curable composition. The cured composition is an adhesive that is suitable for use as a tissue adhesive. In some embodiments, the oxalamido-containing compound in one of the first and second fluids F1, F2 has a molecular weight of at least 250 grams/mole and has at least two oxalamido groups of formula —NR²—(CO)—(CO)—OR¹ wherein R¹ is a hydrocarbyl and wherein R² is hydrogen or hydrocarbyl. The derivatized polyethylene imine in the other of the first and second fluids F1, F2 contains a reaction product of a polyethylene imine with a glycidyl ether. The first seal 112 and the second seal 116 are typically puncturable films and may be multilayer laminates that are welded or otherwise secured to the respective first cylinder 110 and the second cylinder 114. One such construction can be an aluminum foil layer, typically (less than 0.002″) with a High Density Polyethylene (HDPE) layer (also less than 0.002″) that is used to weld to the cylinder seal surface to close to cylinders (i.e., the first cylinder 110 and the second cylinder 114).

In some embodiments, a first viscosity V1 (shown in FIG. 3) of the first fluid F1 is different from a second viscosity V2 of the second fluid F2 (shown in FIG. 3). In some embodiments, a ratio between the second viscosity V2 of the second fluid F2 and the first viscosity V1 of the first fluid F1 is greater than or equal to 2. This means that the second fluid F2 is more viscous than the first fluid F1. The first viscosity V1 of the first fluid F1 and the second viscosity of the second fluid F2 may lie in a range of 5 to 30 Pascals-second. In some embodiments, the first viscosity V1 of the first fluid F1 may be at most 15 Pascals-second. In some embodiments, the second viscosity V2 of the second fluid F2 may be at most 30 Pascals-second. In some embodiments, the first viscosity V1 of the first fluid F1 may be at least 4 Pascals-second. In some embodiments, the second viscosity V2 of the second fluid F2 may be at least 15 Pascals-second.

In some embodiments, the main housing 102 may be a single integral component. However, in some other embodiments, the main housing 102 may include multiple components. For example, the main housing 102 may include a top cover and a bottom cover covering components, such as the first and second cylinders 110, 114. Further, the main housing 102 may include an applicator tip connected to extending from the top cover and the bottom cover proximal to the first end 104. In such cases, the applicator tip may define the applicator outlet 108 disposed at the first end 104. In FIG. 2, the bottom cover of the main housing 102 is removed for illustrative purposes.

The fluid applicator 100 further includes a mixing chamber housing 118 disposed within the main housing 102 and spaced apart from each of the first cylinder 110 and the second cylinder 114 along the longitudinal axis LA.

The fluid applicator 100 further includes a first piercing element 120 substantially aligned with the first seal 112 along the longitudinal axis LA and disposed between the mixing chamber housing 118 and the first cylinder 110. The first piercing element 120 is configured to pierce or puncture the first seal 112 upon application of a force F (shown in FIG. 2). The fluid applicator 100 further includes a second piercing element 122 substantially aligned with the second seal 116 along the longitudinal axis LA and disposed between the mixing chamber housing 118 and the second cylinder 114. The second piercing element 122 is configured to pierce or puncture the second seal 116 upon application of the force F.

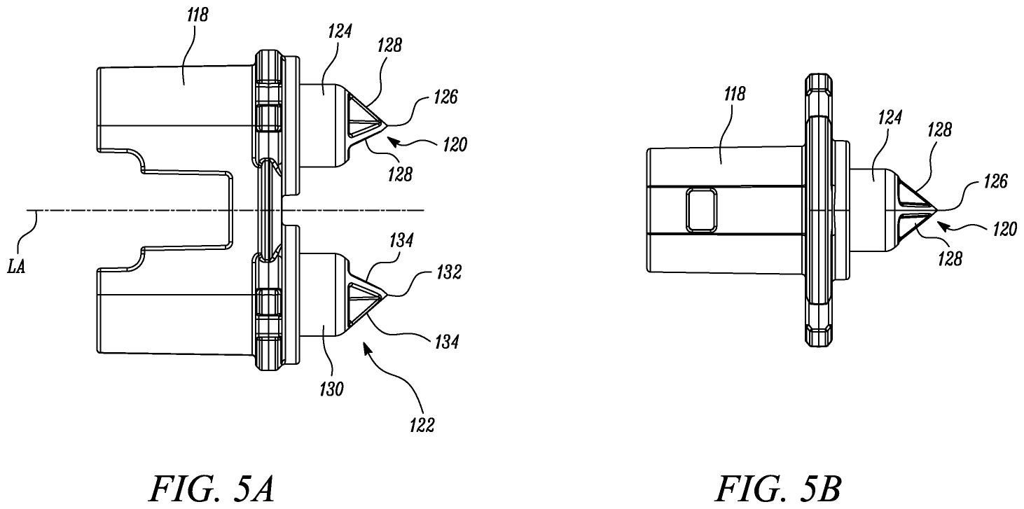

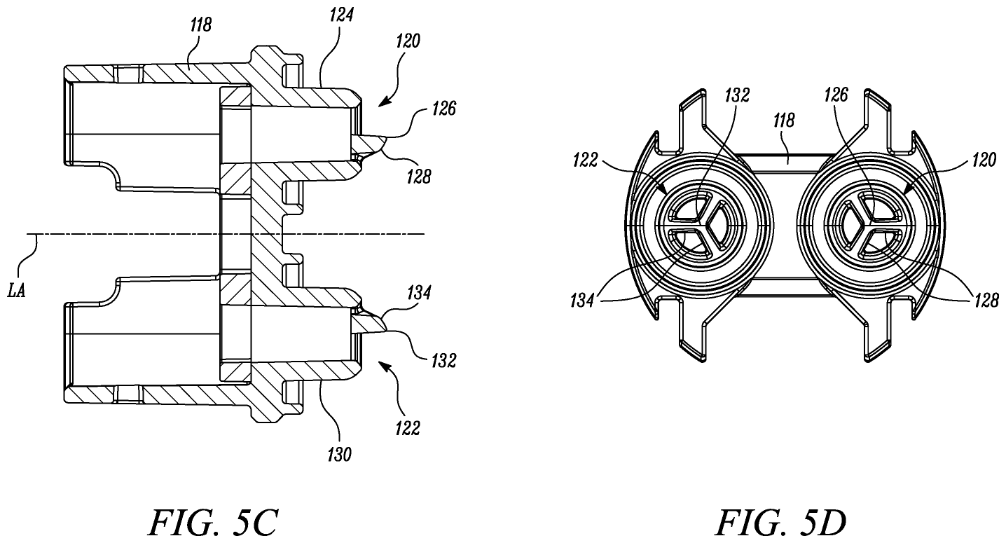

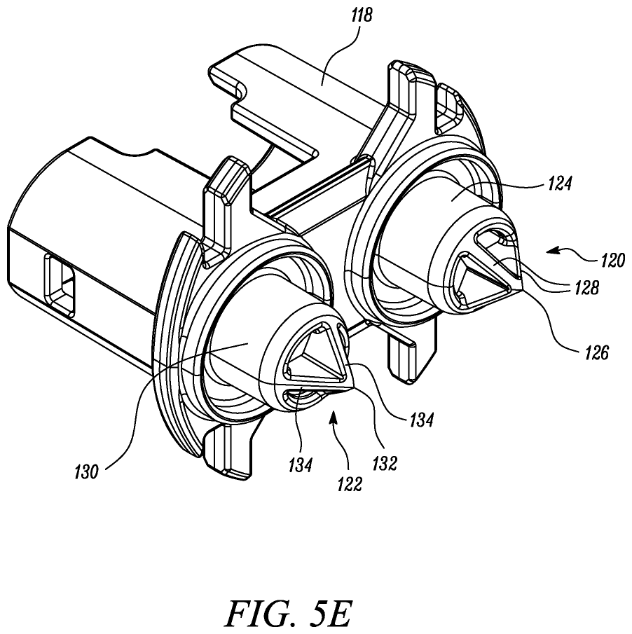

FIG. 5A is a side view of the mixing chamber housing 118, the first piercing element 120, and the second piercing element 122 of the fluid applicator 100 of FIG. 1, according to an embodiment of the present disclosure. FIG. 5B is atop view of the mixing chamber housing 118 and the first piercing element 120, according to an embodiment of the present disclosure. FIG. 5C is a sectional side view of the mixing chamber housing 118, the first piercing element 120, and the second piercing element 122, according to an embodiment of the present disclosure. FIG. 5D is a front view of the mixing chamber housing 118, the first piercing element 120, and the second piercing element 122, according to an embodiment of the present disclosure. FIG. 5E is a perspective front view of the mixing chamber housing 118, the first piercing element 120, and the second piercing element 122, according to an embodiment of the present disclosure.

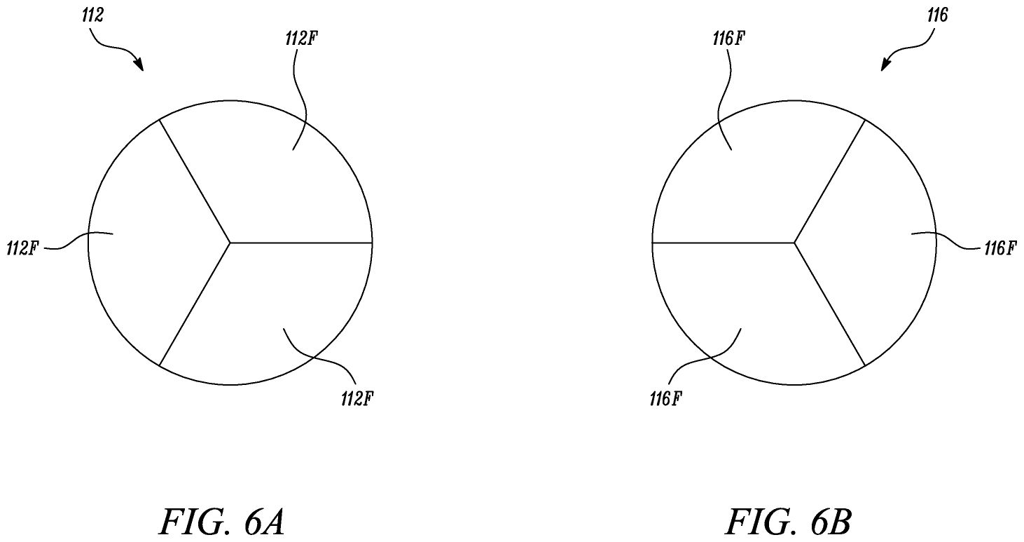

Referring to FIGS. 1 to 5E, the first piercing element 120 includes a first cylindrical base 124 (shown in FIG. 5A) connected to the mixing chamber housing 118 and extending towards the first cylinder 110 (shown in FIG. 2). The first piercing element 120 further includes a first tip 126 (shown in FIG. 5A) spaced apart from the mixing chamber housing 118 and disposed proximal to the first seal 112. The first piercing element 120 further includes a plurality of first walls 128 (shown in FIG. 5A) extending from the first cylindrical base 124 obliquely along the longitudinal axis LA. The plurality of first walls 128 is angularly spaced apart from each other at the first cylindrical base 124 and converge towards each other to intersect at the first tip 126. In some embodiments, the plurality of first walls 128 may be equiangularly arranged about the transverse axis LA. Upon application of a force F (shown in FIG. 2) at the second end 106 of the main housing 102, the first piercing element 120 is configured to pierce the first seal 112. FIG. 6A is a front view of the pierced first seal 112 upon application of the force F at the second end 106 of the main housing 102. Specifically, the first tip 126 engages and pierces the first seal 112 into a plurality of first flaps 112F corresponding to the plurality of first walls 128. In some embodiments, the plurality of first walls 128 is three first walls 128 and the plurality of first flaps 112F is three first flaps 112F corresponding to the three first walls 128. In some other embodiments, the plurality of first walls 128 may be four first walls 128 and the plurality of first flaps 112F may be four first flaps 112F corresponding to the four first walls 128. In some other embodiments, the plurality of first walls 128 may be five first walls 128 and the plurality of first flaps 112F may be five first flaps 112F corresponding to the five first walls 128. In some embodiments, each of the plurality of first walls 128 is substantially triangular.

The second piercing element 122 includes a second cylindrical base 130 (shown in FIG. 5A) connected to the mixing chamber housing 118 and extending towards the second cylinder 114. The second piercing element 122 includes a second tip 132 (shown in FIG. 5A) spaced apart from the mixing chamber housing 118 and disposed proximal to the second seal 116. The second piercing element 122 includes a plurality of second walls 134 (shown in FIG. 5A) extending from the second cylindrical base 130 obliquely along the longitudinal axis LA. The plurality of second walls 134 is angularly spaced apart from each other at the second cylindrical base 130 and converge towards each other to intersect at the second tip 132. In some embodiments, the plurality of second walls 134 may be equiangularly arranged about the transverse axis LA. Upon application of the force F (shown in FIG. 4) at the second end 106 of the main housing 102, the second piercing element 122 is configured to pierce the second seal 116. FIG. 6B is a front view of the pierced second seal 116 upon application of the force F at the second end 106 of the main housing 102. Specifically, the second tip 132 engages and pierces the second seal 116 into a plurality of second flaps 116F corresponding to the plurality of second walls 134. In some embodiments, the plurality of second walls 134 is three second walls 134 and the plurality of second flaps 116F is three second flaps 116F corresponding to the three second walls 134. In some other embodiments, the plurality of second walls 134 may be four second walls 134 and the plurality of second flaps 116F may be four second flaps 116F corresponding to the four second walls 134. In some other embodiments, the plurality of second walls 134 may be five second walls 134 and the plurality of second flaps 116F may be five second flaps 116F corresponding to the five second walls 134. In some embodiments, each of the plurality of second walls 134 is substantially triangular.

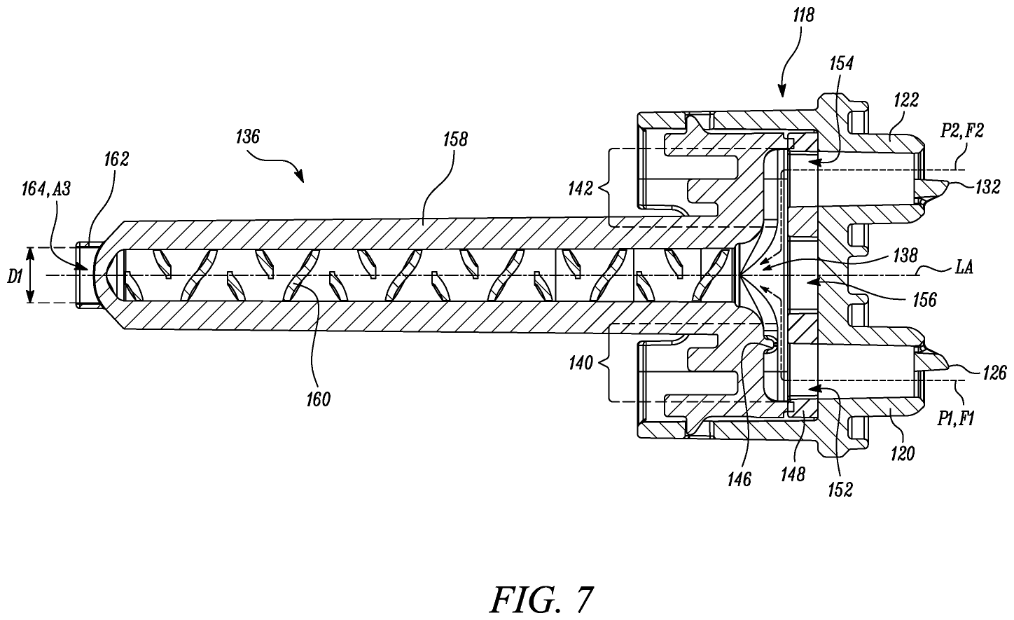

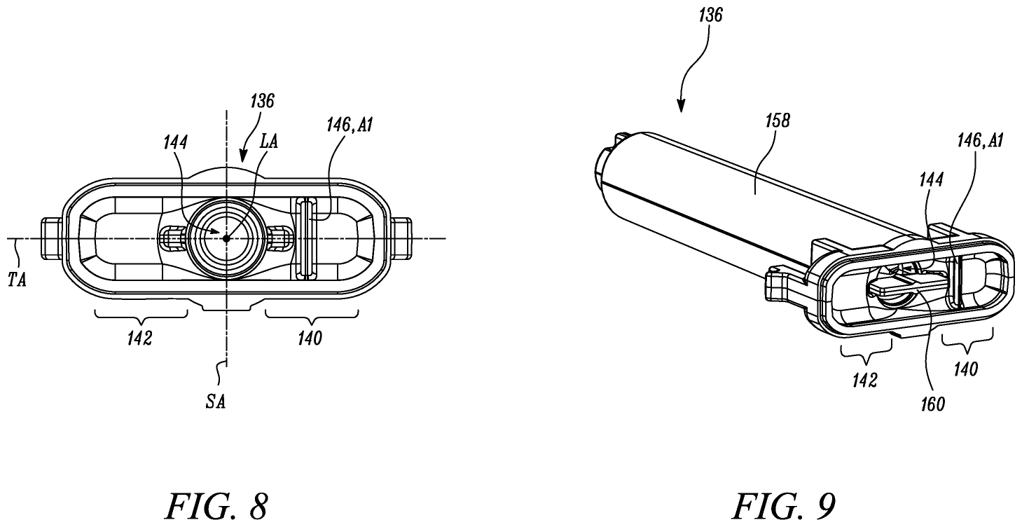

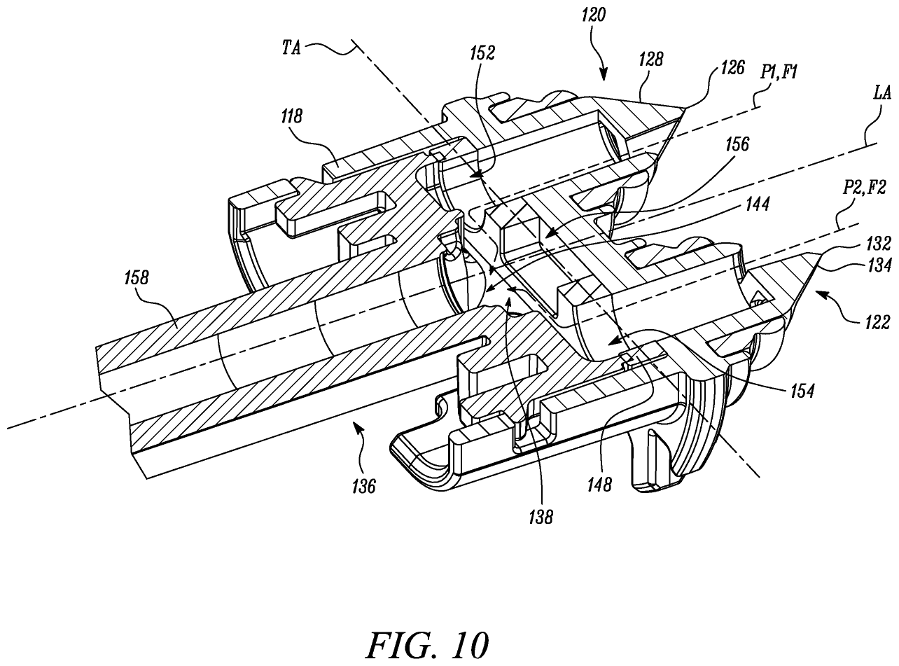

The fluid applicator 100 further includes a nozzle 136 (shown in FIGS. 3 and 4) connected to the mixing chamber housing 118 opposite to each of the first piercing element 120 and the second piercing element 122. FIG. 7 is a sectional side view of the mixing chamber housing 118 and the nozzle 136, according to an embodiment of the present disclosure. FIG. 8 is a front view of the nozzle 136, according to an embodiment of the present disclosure. FIG. 9 is a perspective front view of the nozzle 136, according to an embodiment of the present disclosure. FIG. 10 is a perspective sectional front view of the mixing chamber housing 118 and the nozzle 136, with some components not shown, according to an embodiment of the present disclosure.

Referring to FIGS. 1 to 10, the nozzle 136 extends from the mixing chamber housing 118 to the applicator outlet 108. In some embodiments, the nozzle 136 extends from the mixing chamber housing 118 to the applicator tip of the housing 102 defining the applicator outlet 108. The nozzle 136 and the mixing chamber housing 118 define a mixing chamber cavity 138 (shown in FIG. 10). Upon application of the force F (shown in FIG. 2) at the second end 106 of the main housing 102, the first piercing element 120 engages and pierces the first seal 112, thereby creating a first fluid path P1 (shown in FIG. 10) between the first cylinder 110 and the mixing chamber cavity 138. The first fluid F1 flows through the first fluid path P1 from the first cylinder 110 to the mixing chamber cavity 138. Upon application of the force F (shown in FIG. 2) at the second end 106 of the main housing 102, the second piercing element 122 engages and pierces the second seal 116, thereby creating a second fluid path P2 (shown in FIG. 10) between the second cylinder 114 and the mixing chamber cavity 138. The second fluid F2 flows through the second fluid path P2 from the second cylinder 114 to the mixing chamber cavity 138.

As the first tip 126 engages and pierces the first seal 112 (a seal is heat bonding the foil to the cylinder seal surface) into the plurality of first flaps 112F corresponding to the plurality of first walls 128, the plurality of first flaps 112F remain attached to the first seal 112 of the first cylinder 110. Similarly, as the second tip 132 engages and pierces the second seal 116 into the plurality of second flaps 116F corresponding to the plurality of second walls 134, the plurality of second flaps 116F remain attached to the second seal 116 of the second cylinder 114. In other words, the plurality of first flaps 112F and the plurality of second flaps 116F are not detached from the respective first seal 112 and the second seal 116 upon piercing of the respective first seal 112 and the second seal 116. Due to geometrical design of the first piercing element 120 and the second piercing element 122, the plurality of first flaps 112F remain attached to the first seal 112 of the first cylinder 110, and the plurality of second flaps 116F remain attached to the second seal 116 of the second cylinder 114. Therefore, no part of the foil of the first seal 112 tears away from the first cylinder 110 and no part of the foil of the second seal 116 tears away from the second cylinder 114. In other words, as the foils of the first and second seals 112, 116 are pierced, the foil flaps remain attached to the respective first and second cylinders 110, 114. This may ensure that during dispensing operation by the fluid applicator 100, components, such as the mixing chamber cavity 138, the nozzle 136, and/or the applicator tip of the housing 102 defining the applicator outlet 108, may not be blocked by any torn away pieces of the first seal 112 and the second seal 116 which was otherwise a challenge in the conventional fluid applicators/dispensers.

In some embodiments, the mixing chamber housing 118 and the nozzle 136 further define a first portion 140 (shown in FIG. 7) substantially aligned with the first piercing element 120 along the longitudinal axis LA. The first fluid F1 (shown in FIG. 3) flows through the first fluid path P1 (shown in FIG. 3) from the first cylinder 110 to the first portion 140. The mixing chamber housing 118 and the nozzle 136 further define a second portion 142 substantially aligned with the second piercing element 122 along the longitudinal axis LA. The second fluid F2 (shown in FIG. 3) flows through the second fluid path P2 (shown in FIG. 3) from the second cylinder 114 to the second portion 142.

The mixing chamber housing 118 and the nozzle 136 further define a mixing through aperture 144 (shown in FIGS. 8 to 10) in fluid communication with the mixing chamber cavity 138 disposed between the first portion 140 and the second portion 142 along a transverse axis TA orthogonal to the longitudinal axis LA. The first fluid F1 flows through the first portion 140 to the mixing through aperture 144. The second fluid F2 flows through the second portion 142 to the mixing through aperture 144.

In some embodiments, the mixing chamber housing 118 and the nozzle 136 further define a flow restrictor 146 (shown in FIGS. 8 and 9) disposed on the nozzle 136 and between the first portion 140 and the mixing through aperture 144 along the transverse axis TA and extending along a sagittal axis SA orthogonal to each of the transverse axis TA and the longitudinal axis LA, such that the flow restrictor 146 restricts the flow of the first fluid F1 (shown in FIG. 7) from the first portion 140 to the mixing through aperture 144. In some embodiments, the flow restrictor 146 blocks a cross-sectional area A1 in a plane defined by the sagittal axis SA and the longitudinal axis LA by greater than or equal to about 20% and less than or equal to about 80% to restrict the flow of the first fluid F1 from the first portion 140 to the mixing through aperture 144. The cross-sectional area A1 is therefore defined in the SA-LA plane. In other embodiments, the flow restrictor 146 blocks the cross-sectional area A1 by greater than or equal to about 30% and less than or equal to about 70% to restrict the flow of the first fluid F1 from the first portion 140 to the mixing through aperture 144. In other embodiments, the flow restrictor 146 blocks the cross-sectional area A1 by about 50% to restrict the flow of the first fluid F1 from the first portion 140 to the mixing through aperture 144.

The flow restrictor 146 may slow down an initial dispense of the first fluid F1 (i.e., the fluid having a lower viscosity). For a desirable tensile strength of the initial portion of the dispense bead, it is required to limit the flow rate of the first fluid F1 because of the relatively lower first viscosity V1 (shown in FIG. 7) of the first fluid F1 as compared to the second viscosity V2 (shown in FIG. 7) of the second fluid F2. Hence, the flow restrictor 146 limits or restricts the flow of the first fluid F1 from the first portion 140 to the mixing through aperture 144 and may therefore improve the tensile strength of the initial portion of the dispense bead formed by even mixing of the first fluid F1 and the second fluid F2.

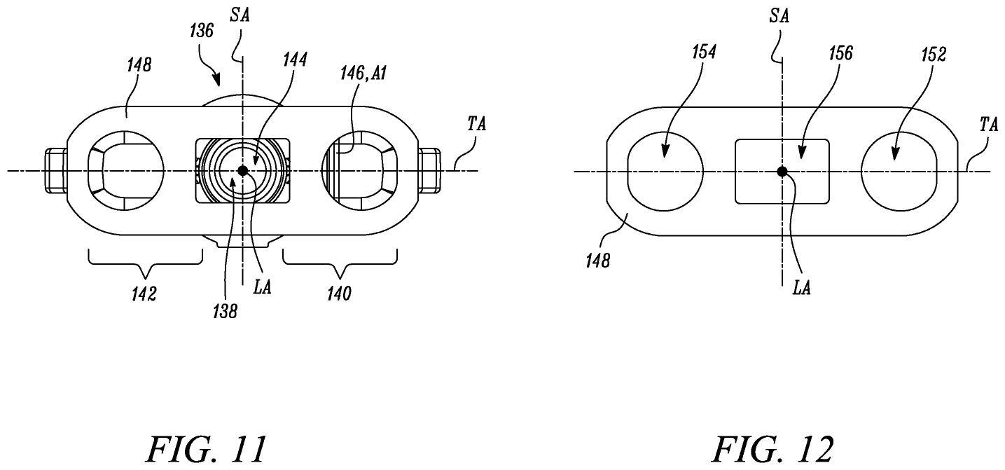

In some embodiments, the fluid applicator 100 further includes a nozzle gasket 148 (shown in FIGS. 7 and 10) disposed inside the mixing chamber housing 118 and forming a sealing contact between the mixing chamber housing 118 and the nozzle 136. In other words, the nozzle gasket 148 may provide a leak proof attachment of the nozzle 136 to the mixing chamber housing 118. FIG. 11 is a front view of the nozzle 136 and the nozzle gasket 148 of the fluid applicator 100 of FIG. 1, with other components not shown, according to an embodiment of the present disclosure. FIG. 12 is a front view of the nozzle gasket 148, according to an embodiment of the present disclosure. In such embodiments, the nozzle 136, the mixing chamber housing 118, and the nozzle gasket 148 define the mixing chamber cavity 138.

With reference to FIGS. 7 to 12, the nozzle gasket 148 defines a first through aperture 152 in fluid communication with the first fluid path P1. The nozzle gasket 148 further defines a second through aperture 154 in fluid communication with the second fluid path P2. In other words, the first through aperture 152 of the nozzle gasket 148 provides input from the first cylinder 110 and the second through aperture 154 of the nozzle gasket 148 provides input from the second cylinder 114.

The nozzle gasket 148 further defines a third through aperture 156 disposed between the first through aperture 152 and the second through aperture 154 along the transverse axis TA, such that a portion of at least one of the first and second fluids F1, F2 which flows from the respective first and second fluid paths P1, P2 is trapped within the third through aperture 156. Specifically, a portion of at least one of the first and second fluids F1, F2 which flows from the respective first and second fluid paths P1, P2 is trapped within the third through aperture 156. More specifically, the portion of the at least one of the first and second fluids F1, F2 flowing from the respective first and second fluid paths P1, P2 flows in an opposite direction of the flow from the mixing chamber cavity 138 into the nozzle 136 to get trapped within the third through aperture 156.

Therefore, the third through aperture 156 catches or traps the initial flow of dispense when the first fluid F1 (i.e., the fluid having lower viscosity) initially leads the second fluid F2 (i.e., the fluid having higher viscosity) upon activation.

Thus, the third through aperture 156 acts as a fluid trap to capture the initial dispense bead (i.e., the portion of the at least one of the first and second fluids F1, F2 which flows from the respective first and second fluid paths P1, P2). In some embodiments, a volume of the portion of the at least one of the first and second fluids F1, F2 trapped within the third through aperture 156 is greater than or equal to about 5 mm³ and less than or equal to about 25 mm³.

In some embodiments, the nozzle 136 further includes a nozzle body 158 in fluid communication with the mixing chamber cavity 138. In some embodiments, the nozzle 136 further includes a static mixer 160 disposed inside the nozzle body 158. The static mixer 160 is not shown in FIGS. 8 and 10 for illustrative purposes only. The static mixer 160 mixes of the first fluid F1 and the second fluid F2 prior to delivery of the dispense bead via the applicator outlet 108.

The inclusion of the third through aperture 156 in the nozzle gasket 148 may remove an initial material (e.g., the initial portion of the first fluid F1) that could be directed to the static mixer 160 in the nozzle 136. Therefore, the end quality of product dispense through the applicator outlet 108 may be good as the initial material that cannot be mixed is trapped. This may further improve the tensile strength of the dispense bead formed by mixing of the first fluid F1 and the second fluid F2.

The nozzle 136 further includes a nozzle tip 162 defining a nozzle outlet 164 for dispensing at least one of the first and second fluids F1, F2 from the nozzle 136. The nozzle tip 162 is connected to and extends from the nozzle body 158. The nozzle outlet 164 may have a substantially circular cross-section. In some embodiments, the nozzle outlet 164 may have a substantially rectangular cross-section.

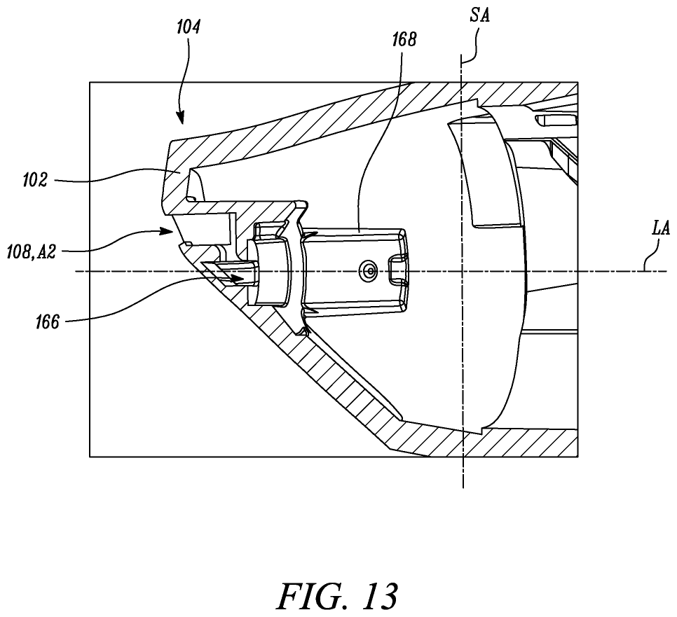

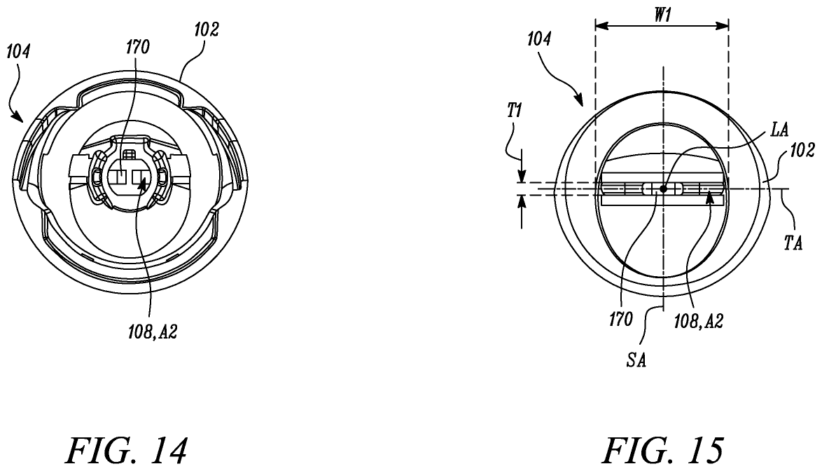

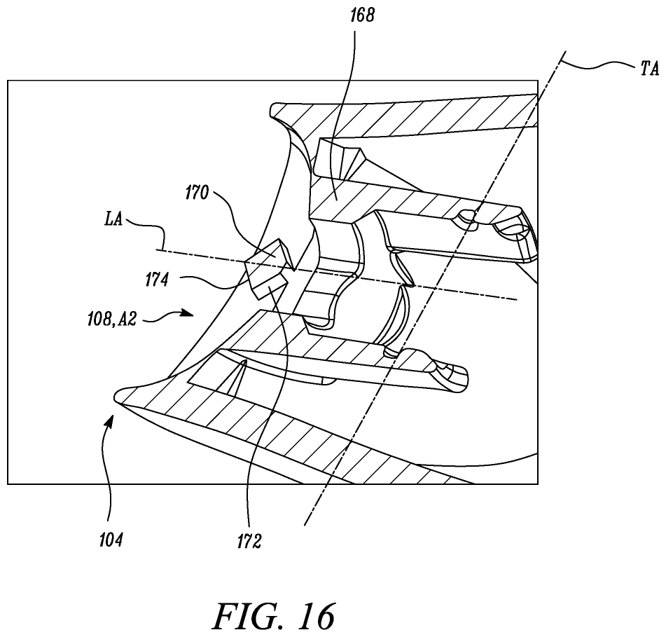

FIG. 13 is a cross-sectional view of the first end 104 of the fluid applicator 102 of FIG. 1, according to an embodiment of the present disclosure. Specifically, FIG. 13 is the cross-sectional view of the first end 104 of the fluid applicator 102 in a plane defined by the longitudinal axis LA and the sagittal axis SA. FIG. 14 is a front view of the first end 104 of the fluid applicator 102, according to an embodiment of the present disclosure. FIG. 15 is a rear view of the first end 104 of the fluid applicator 102, according to an embodiment of the present disclosure. FIG. 16 is another cross-sectional view of the first end 104 of the fluid applicator 102 of FIG. 1, according to an embodiment of the present disclosure. Specifically, FIG. 16 is the cross-sectional view of the first end 104 of the fluid applicator 102 in a plane defined by the longitudinal axis LA and the transverse axis TA.

Referring to FIG. 13, in some embodiments, the main housing 102 includes a capture well 166 adjacent the applicator outlet 108, such that a portion of at least one of the first and second fluids F1, F2 (shown in FIG. 3) which flows from the nozzle outlet 164 (shown in FIG. 7) is trapped within the capture well 166. In some embodiments, the applicator tip of the main housing 102 includes the capture well 166. In some embodiments, the portion of the at least one of the first and second fluids F1, F2 which is trapped within the capture well 166 is greater than or equal to about 2% and less than or equal to about 15% of the total volume of the first and second fluids F1, F2 to be dispensed from the nozzle outlet 164. In some embodiments, the portion of the at least one of the first and second fluids F1, F2 which is trapped within the capture well 166 is about 5% of the total volume of the first and second fluids F1, F2 to be dispensed from the nozzle outlet 164.

The capture well 166 acts as a fluid trap to capture the initial bolus of a mixture of the first fluid F1 and the second fluid F2 after being mixed within the mixing chamber cavity 138 and passing though the nozzle body 158. Therefore, a desirable tensile strength of the dispense bead may be obtained by capturing the portion of the at least one of the first and second fluids F1, F2 within the capture well 166.

As shown in FIGS. 14 and 15, in some embodiments, the applicator outlet 108 has a substantially rectangular cross-section or an elliptical cross-section. In other embodiments, the applicator outlet 108 may have a substantially circular cross-section.

In some embodiments, the applicator outlet 108 includes a maximum outlet width W1 (shown in FIG. 15) along the transverse axis TA orthogonal to the longitudinal axis LA and a maximum outlet thickness T1 (shown in FIG. 15) orthogonal to each of the transverse axis TA and the longitudinal axis LA. The maximum outlet width W1 is greater than the maximum outlet thickness T1 by a factor of at least 3. In some embodiments, the maximum outlet width W1 may be greater than the maximum outlet thickness T1 by a factor of at least 4, or at least 5.

Such dimensions of the maximum outlet width W1 and the maximum outlet thickness T1 of the applicator outlet 108 may provide a desirable height and width of the dispense bead formed by mixing the first fluid F1 and the second fluid F2. Such dimensions of the applicator outlet 108 may cause the dispensed bead to achieve a desirable profile shape after exiting through the nozzle 136.

In some embodiments, a ratio between the maximum outlet width W1 of the applicator outlet 108 and a maximum outlet width D1 (shown in FIG. 7) of the nozzle outlet 164 is greater than or equal to about 2 and less than or equal to about 5. In other embodiments, the ratio between the maximum outlet width W1 of the applicator outlet 108 and the maximum outlet width D1 of the nozzle outlet 164 is about 3.8. In some embodiments, a ratio between an applicator exit area A2 of the applicator outlet 108 and a nozzle exit area A3 (shown in FIG. 7) of the nozzle outlet 164 is greater than or equal to about 5 and less than or equal to about 20. In other embodiments, the ratio between the applicator exit area A2 of the applicator outlet 108 and the nozzle exit area A3 of the nozzle outlet 164 is about 14.44.

In some embodiments, the main housing 102 further includes an outlet interface 168 (shown in FIGS. 13 and 16) proximal to the applicator outlet 108. In some embodiments, the applicator tip of the main housing 102 includes the outlet interface 168. The nozzle tip 162 (shown in FIG. 7) is configured to be coupled to the outlet interface 168. In this way, the nozzle 136 is fluidly communicated with the applicator outlet 108.

In some embodiments, the outlet interface 168 includes a fluid separator 170 (shown in FIGS. 14 to 16) disposed between the nozzle outlet 164 and the applicator outlet 108. At least a portion 172 (shown in FIG. 16) of the fluid separator 170 proximal to the nozzle outlet 164 tapers outwardly relative to the longitudinal axis LA in a flow direction of at least one of the first and second fluids F1, F2 (shown in FIG. 3) such that the flow of the at least one of the first and second fluids F1, F2 from the nozzle outlet 164 is directed outward towards edges of the applicator outlet 108. In some embodiments, the remaining portion 174 of the fluid separator 170 proximal to the applicator outlet 108 tapers inwardly relative to the longitudinal axis LA in the flow direction of the at least one of the first and second fluids F1, F2 such that the flow of the at least one of the first and second fluids F1, F2 from the nozzle outlet 164 is directed inward towards the center of the applicator outlet 108.

In some embodiments, the portion 172 of the fluid separator 170 tapers outwardly such that a flow of a mixture of the first and second fluids F1, F2 from the nozzle outlet 164 is directed outward towards edges of the applicator outlet 108. In some embodiments, the remaining portion 174 of the fluid separator 170 tapers inwardly such that the flow of the mixture of the first and second fluids F1, F2 from the nozzle outlet 164 is directed inward towards the center of the applicator outlet 108.

Therefore, the fluid separator 170 directs the flow of the at least one of the first and second fluids F1, F2 or the mixture of the first and second fluids F1, F2 from the nozzle outlet 164 towards the edges as well as the center of the applicator outlet 108. This direction of the flow of the at least one of the first and second fluids F1, F2 from the nozzle outlet 164 to the applicator outlet 108 may ensure that the at least one of the first and second fluids F1, F2 is spread uniformly and may provide a desirable height and width of the dispense bead that exits out of the applicator outlet 108.

In some embodiments, the fluid applicator 100 according to the present disclosure may include the outlet interface 168 including the fluid separator 170 (shown in FIG. 15) as well as the capture well 166 (shown in FIG. 13) adjacent the applicator outlet 108. In some embodiments, the fluid applicator 100 may include the fluid separator 170 as well as the applicator outlet 108 having the maximum outlet width W1 greater than the maximum outlet thickness T1 by the factor of at least 3 (shown in FIG. 15). In some embodiments, the fluid applicator 100 according to the present disclosure may include the fluid separator 170, the capture well 166, and the applicator outlet 108 as shown in FIG. 15.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A fluid applicator comprising:
a main housing comprising a first end and a second end opposite to the first end, the main housing extending along a longitudinal axis defined between the first end and the second end, wherein the main housing defines an applicator outlet disposed at the first end;
a first cylinder disposed within the main housing and comprising a first seal for holding a first fluid, wherein at least 85% of a total volume of the first cylinder is filled with the first fluid;
a second cylinder spaced apart from the first cylinder and disposed within the main housing, the second cylinder comprising a second seal for holding a second fluid different from the first fluid, wherein at least 85% of a total volume of the second cylinder is filled with the second fluid;
a mixing chamber housing disposed within the main housing and spaced apart from each of the first cylinder and the second cylinder along the longitudinal axis;
a first piercing element substantially aligned with the first seal along the longitudinal axis and disposed between the mixing chamber housing and the first cylinder, the first piercing element comprising a first cylindrical base connected to the mixing chamber housing and extending towards the first cylinder, a first tip spaced apart from the mixing chamber housing and disposed proximal to the first seal, and a plurality of first walls extending from the first cylindrical base obliquely along the longitudinal axis, the plurality of first walls being angularly spaced apart from each other at the first cylindrical base and converging towards each other to intersect at the first tip;
a second piercing element substantially aligned with the second seal along the longitudinal axis and disposed between the mixing chamber housing and the second cylinder, the second piercing element comprising a second cylindrical base connected to the mixing chamber housing and extending towards the second cylinder, a second tip spaced apart from the mixing chamber housing and disposed proximal to the second seal, and a plurality of second walls extending from the second cylindrical base obliquely along the longitudinal axis, the plurality of second walls being angularly spaced apart from each other at the second cylindrical base and converging towards each other to intersect at the second tip; and
a nozzle connected to the mixing chamber housing opposite to each of the first piercing element and the second piercing element, the nozzle extending from the mixing chamber housing to the applicator outlet, wherein the nozzle and the mixing chamber housing define a mixing chamber cavity;
wherein, upon application of a force at the second end of the main housing:
the first tip engages and pierces the first seal into a plurality of first flaps corresponding to the plurality of first walls, thereby creating a first fluid path between the first cylinder and the mixing chamber cavity, wherein the first fluid flows through the first fluid path from the first cylinder to the mixing chamber cavity; and
the second tip engages and pierces the second seal into a plurality of second flaps corresponding to the plurality of second walls, thereby creating a second fluid path between the second cylinder and the mixing chamber cavity, wherein the second fluid flows through the second fluid path from the second cylinder to the mixing chamber cavity.

2. The fluid applicator of claim 1, wherein a first viscosity of the first fluid is different from a second viscosity of the second fluid.

3. The fluid applicator of claim 2, wherein a ratio between the second viscosity of the second fluid and the first viscosity of the first fluid is greater than or equal to 2.

4. The fluid applicator of claim 1, wherein:
the plurality of first walls is three first walls and the plurality of first flaps is three first flaps corresponding to the three first walls; and
the plurality of second walls is three second walls and the plurality of second flaps is three second flaps corresponding to the three second walls.

5. The fluid applicator of claim 1, wherein the mixing chamber housing and the nozzle further define:
a first portion substantially aligned with the first piercing element along the longitudinal axis, wherein the first fluid flows through the first fluid path from the first cylinder to the first portion;
a second portion substantially aligned with the second piercing element along the longitudinal axis, wherein the second fluid flows through the second fluid path from the second cylinder to the second portion;
a mixing through aperture in fluid communication with the mixing chamber cavity disposed between the first portion and the second portion along a transverse axis orthogonal to the longitudinal axis, wherein the first fluid flows through the first portion to the mixing through aperture; and wherein the second fluid flows through the second portion to the mixing through aperture; and
a flow restrictor disposed on the nozzle and between the first portion and the mixing through aperture along the transverse axis and extending along a sagittal axis orthogonal to each of the transverse axis and the longitudinal axis, such that the flow restrictor restricts the flow of the first fluid from the first portion to the mixing through aperture.

6. The fluid applicator of claim 5, wherein the flow restrictor blocks a cross-sectional area in a plane defined by the sagittal axis and the longitudinal axis by greater than or equal to about 20% and less than or equal to about 80% to restrict the flow of the first fluid from the first portion to the mixing through aperture.

7. The fluid applicator of claim 1, further comprising a nozzle gasket disposed inside the mixing chamber housing and forming a sealing contact between the mixing chamber housing and the nozzle, the nozzle gasket defining:
a first through aperture in fluid communication with the first fluid path;
a second through aperture in fluid communication with the second fluid path; and
a third through aperture disposed between the first through aperture and the second through aperture along a transverse axis perpendicular to the longitudinal axis, such that a portion of at least one of the first and second fluids which flows from the respective first and second fluid paths is trapped within the third through aperture.

8. The fluid applicator of claim 7, wherein a volume of the portion of the at least one of the first and second fluids trapped within the third through aperture is greater than or equal to about 5 mm³ and less than or equal to about 25 mm³.

9. The fluid applicator of claim 1, wherein the nozzle further comprises:
a nozzle body in fluid communication with the mixing chamber cavity;
a static mixer disposed inside the nozzle body; and
a nozzle tip defining a nozzle outlet for dispensing at least one of the first and second fluids from the nozzle, the nozzle tip connected to and extending from the nozzle body, wherein the nozzle outlet has a substantially circular cross-section.

10. The fluid applicator of claim 9, wherein the main housing comprises a capture well adjacent the applicator outlet, such that a portion of at least one of the first and second fluids which flows from the nozzle outlet is trapped within the capture well.

11. The fluid applicator of claim 10, wherein the portion of the at least one of the first and second fluids which is trapped within the capture well is greater than or equal to about 2% and less than or equal to about 15% of the total volume of the first and second fluids to be dispensed from the nozzle outlet.

12. The fluid applicator of claim 1, wherein the applicator outlet has a substantially rectangular cross-section or an elliptical cross-section.

13. The fluid applicator of claim 9, wherein the applicator outlet comprises a maximum outlet width along a transverse axis orthogonal to the longitudinal axis and a maximum outlet thickness orthogonal to each of the transverse axis and the longitudinal axis, and wherein the maximum outlet width is greater than the maximum outlet thickness by a factor of at least 3.

14. The fluid applicator of claim 13, wherein a ratio between the maximum outlet width of the applicator outlet and a maximum outlet width of the nozzle outlet is greater than or equal to about 2 and less than or equal to about 5.

15. The fluid applicator of claim 9, wherein a ratio between an applicator exit area of the applicator outlet and a nozzle exit area of the nozzle outlet is greater than or equal to about 5 and less than or equal to about 20.

16. The fluid applicator of claim 9, wherein the main housing further comprises an outlet interface proximal to the applicator outlet, and wherein the nozzle tip is configured to be coupled to the outlet interface.

17. The fluid applicator of claim 16, wherein the outlet interface comprises a fluid separator disposed between the nozzle outlet and the applicator outlet, wherein at least a portion of the fluid separator proximal to the nozzle outlet tapers outwardly relative to the longitudinal axis in a flow direction of at least one of the first and second fluids such that the flow of the at least one of the first and second fluids from the nozzle outlet is directed outward towards edges of the applicator outlet.

18. The fluid applicator of claim 17, wherein a remaining portion of the fluid separator proximal to the applicator outlet tapers inwardly relative to the longitudinal axis in the flow direction of the at least one of the first and second fluids such that the flow of the at least one of the first and second fluids from the nozzle outlet is directed inward towards a center of the applicator outlet.

19. The fluid applicator of claim 1, wherein each of the plurality of first walls is substantially triangular.

20. The fluid applicator of claim 1, wherein each of the plurality of second walls is substantially triangular.

21-61. (canceled)
